(19) United States
(12) Patent Application Publication
(10) Pub. No.: US 2018/0085653 A1
(43) Pub. Date: Mar. 29, 2018

(54) Athletic Tracking Device

(71) Applicant: Melissa Picker, St. James, MO (US)

(72) Inventor: Melissa Picker, St. James, MO (US)

(21) Appl. No.: 15/716,090

(22) Filed: Sep. 26, 2017

Related U.S. Application Data
(60) Provisional application No. 62/400,089, filed on Sep. 26, 2016.

(51) Int. Cl. A63B 71/06; A63B 24/00; G09B 19/00; G09B 5/02; A61B 5/024; A61B 5/00

(52) U.S. Cl. CPC A63B 71/0622; G06K 19/06028; G09B 19/0092; G09B 5/02; A63B 71/0686; A61B 5/02438; A61B 5/681; A63B 2071/0625; A63B 2071/0663; A63B 2220/17; A63B 2230/06; A63B 2220/73; A63B 2225/20; A63B 2071/0655; A63B 24/0062

(57) ABSTRACT

An activity tracking device having at least two different models. One model would be a fitness smart watch version, with or without extra sliders, buttons or watch facing touch screen selection for tracking water and fruit/vegetable intakes, and with the typical smart watch features, including a pedometer, heart-rate monitor, altimeter, stop watch, vibrating notifications, alerts, etc. The second model would have no typical watch functionality, but, would have only a way of counting simple health goals, such as the number of glasses of water one drinks, with a slider or counter button; however, users would choose an appropriate band for tracking their goals for various activities like drinking enough water, fruit and vegetable intake, daily exercises, meditation breaks during the day and more.

### CROSS-REFERENCE TO RELATED APPLICATION

The present application is related to and claims priority from prior provisional application Ser. No. 62/400,089, filed Sep. 26, 2016 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. Field of the Invention

The present invention relates generally to the field of athletic tracking devices and more specifically relates to an activity tracking device having at least two different models. One model would be a fitness smart watch version, with or without extra sliders, buttons or a watch facing touch screen selection, for tracking water and fruit/vegetable intakes, and with the typical smart watch features, including a pedometer, heart-rate monitor, altimeter, stop watch, vibrating notifications, alerts, etc. The second model is a simple band version that would have no automated tracking; however, users would choose an appropriate band for tracking their goals for various activities like drinking enough water, fruit and vegetable intake, daily exercises, meditation breaks during the day and more.

### 2. Description of the Related Art

Fitbits are activity trackers, wireless-enabled wearable technology devices that measure data such as the number of steps walked, heart rate, quality of sleep, steps climbed, and other personal metrics. The first of these was the Fitbit Tracker. Fitbits and other types of sport/fit watches allow users to set personal goals, but this is not enough motivation to help some users stay on track to accomplish their goals. Therefore, a need exists for a specially designed new kind of fitness tracker that will allow users to have personally chosen inspirational pictures, like a beloved child or beautiful beach to provide the motivation to reach goals for oneself. In addition, self-recorded, inspirational short videos (flicks or personalized snapchats) would also provide motivation. Individualized mantras and voice text messages can be used to enhance their motivation as well. A virtual track or trail can be displayed on the watch face to provide motivation display in proportion where the user is relative to their goal. Other fitness trackers require the users to log into the related App to log other important health data, like water, food, and other health goals, however, this version will allow the user to quickly log this data directly on the watch itself without having to log into the app.

Various attempts have been made to solve the above-mentioned problems such as those found in U.S. Pat. and Pub. Nos. 2016/0077492 to Brown et al.; 2014/0099614 to Hu et al.; U.S. Pat. No. 9,050,488 to Brumback et al.; 2015/0066172 to Chiang Ying Yi and U.S. Pat. No. 8,517,896 to Robinette et al. This art is representative of athletic tracking and goal incentivized devices. None of the above inventions and patents, taken either singly or in combination, is seen to describe the invention as claimed.

Ideally, an athletic tracking device should provide a way to track fitness goals and provide motivation for reaching those goals and, yet would operate reliably and be manufactured at a modest expense. Thus, a need exists for a reliable athletic tracking device with motivational features to achieve the desired results.

### BRIEF SUMMARY OF THE INVENTION

In view of the foregoing disadvantages inherent in the known art, the present invention provides a novel athletic tracking device having motivational features. The general purpose of the present invention, which will be described subsequently in greater detail is to provide an athletic tracking device having at least two different models and also having motivational features. One model would be a fitness smart watch version, with or without extra sliders, buttons or watch facing touch screen selection for tracking water and fruit/vegetable intakes, and with the typical smart watch features, including a pedometer, heart-rate monitor, altimeter, stop watch, vibrating notifications, alerts, etc. Another option, depending on the design is to use a touchscreen on the face of the watch version to increase the number of glasses of water consumed, etc.

The second model is a simple band version that would have no typical watch functionality, but, would have only a way of counting simple health goals, such as the number of glasses of water one drinks, with a slider or counter button; however, users would choose an appropriate band for tracking their goals for various activities like drinking enough water, fruit and vegetable intake, daily exercises, meditation breaks during the day and more. The slider can be reset to zero by sliding it back down to zero. The counter button could be reset to zero by holding it down for a short time period or possibly via an app where the counter data may be loaded.

The unique features of this invention will provide the following benefits for consumers everywhere:
Fitness bands and watches that can be customized, using pictures, short inspirational self-recorded videos, voice messages and mantras, to provide individualized motivation and to share as motivation with friends. Individuals will find the power to reach their goal in order to reveal and share their personal “Power Pic” (children, mother or father) for whom they reach their goal in honor of each day.
The sliding trackers or buttons or watch facing touch screen selection provides a quick, easy and customizable way for users to track what is important to them each day without having to write it down on a piece of paper or use some kind of complicated app on their phones. It's much quicker to slide a tracker up a notch multiple times day than to log into a phone app and manually log something over and over again. It's much easier to log a goal directly on the watch via a button or touch screen selection than logging into an app as well.
The power to reveal photos, videos, voice messages when goals are achieved will enhance the motivational levels of users
The ability to choose a custom “voltage level” or the level of daily automated motivation in the form of photos, videos, and messages
The ability to request a “power surge” and see a photo or a video, or to hear a voice message when a person needs extra inspiration
A “power grid of friends” to send/receive motivational photos, videos and voice messages to inspire friends as well
A “power grid of community sponsors” that can become involved by providing motivational support to encourage a healthy lifestyle within their communities
A “fundraising power grid” to provide many community fund-raising possibilities
Sliders, buttons or watch facing touchscreen selection, for tracking the number of glasses of water and the number of fruits and vegetables that are eaten, would be available on certain versions of the watch.
A button on the watch can be used for viewing the number of steps, elevation, heart rate, and other smart watch items
The date and time are displayed on the watch version
The watch version would notify users automatically when they reach their goals via a sound or beep and/or having the watch vibrate. The watch version would have all the functionality expected for smart watches, include access to text messages, reminders to keep moving, notification of phone calls, etc.
It provides friend/family/team/group healthy lifestyle motivation by sharing and sending motivational photos, recorded videos and voice message
It can be a community teaching tool as well
The watch/app can be a teaching/learning tool as it could send out automatic tips and health advice based on app selections (running/walking/yoga/healthy eating/) as the user progresses towards a goal
Notifications can be turned on or off and set for once up to several times a day
A watch version may display a virtual track on the watch face showing where the user currently is in relation to their goal for motivation
The simplest version provides the user the simplest way of counting and tracking their health goals by just moving a slider or pressing a counter button. This data could be loaded to an app. Resetting the counters to zero by adjusting the slider or holding down the button for a short period of time is also extremely simple. The simplest version could be tied to an app for loading the counter information and resetting of the counters at a specific time each day as well. The user can track any goals one wants with this simple band with a counter.

The present invention holds significant improvements and serves as an improved athletic tracking device with motivational features. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, the Athletic Tracking Device is constructed and operative according to the teachings of the present invention.

FIG. 1 shows a perspective view illustrating an athletic tracking device with the handle member up and showing an inspirational photograph according to an embodiment of the present invention.

FIG. 2 is a perspective view illustrating an athletic tracking device with the handle member down and showing a different motivational image according to an embodiment of the present invention of FIG. 1.

FIG. 3 is a perspective view illustrating the watch version of an athletic tracking device showing various monitors according to an embodiment of the present invention of FIG. 1.

FIG. 4 is a perspective view illustrating a close up of the band version of an athletic tracking device according to an embodiment of the present invention of FIG. 1.

FIG. 5 is a side view of an athletic tracking device according to an embodiment of the present invention of FIG. 1.

The various embodiments of the present invention will hereinafter be described in conjunction with the appended drawings, wherein like designations denote like elements.

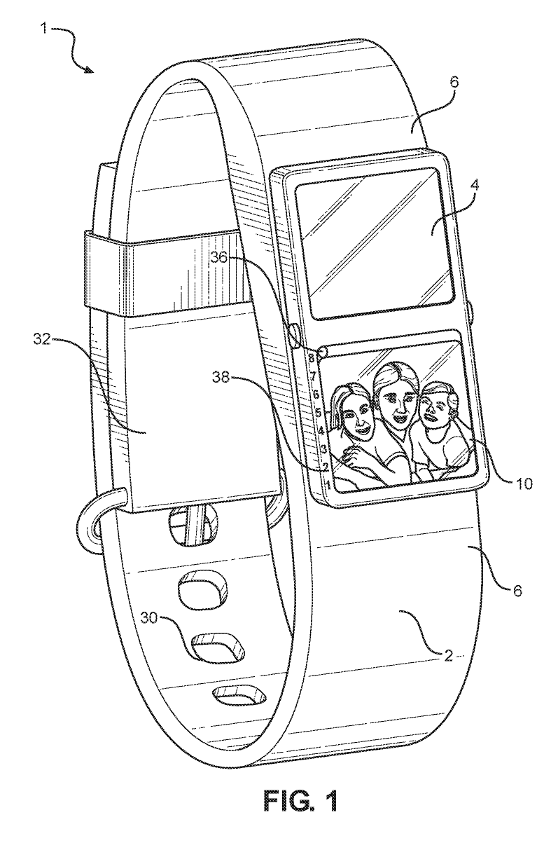

Referring now to the drawings by numerals of reference there is shown in FIGS. 1-5, the athletic tracking device 1 is preferably manufactured in several different versions of fitness watches and bands available to accommodate individual needs and budgets. Both the watch versions and bands would have adjustable clasps so they will fit securely around a person's wrist and they will be available in a variety of colors and color combinations to appeal to users. As discussed above, embodiments of the present invention relate to an athletic tracking device 1 having at least two different models as used to improve the tracking of athletic progress.

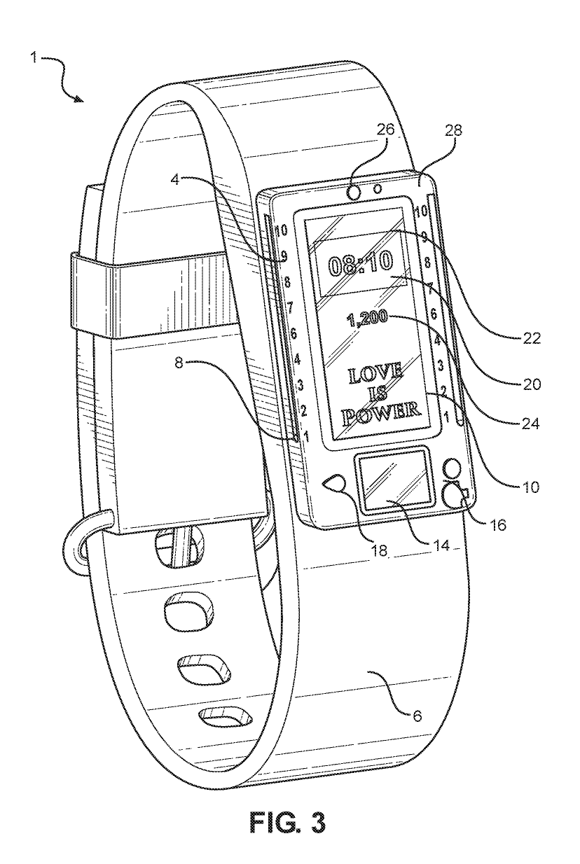

The Power Pic Set Up: For the watch version of the athletic tracking device 1, as best seen in FIG. 3, the images 10 including Power pics (photos), the inspirational videos (flicks), and voice messages would be recorded and uploaded via the app linked to the watch. Individuals could record and upload a short video for themselves through the app, which will be displayed when reaching their goals. In addition, typed messages could be created and uploaded via the app for display as well.

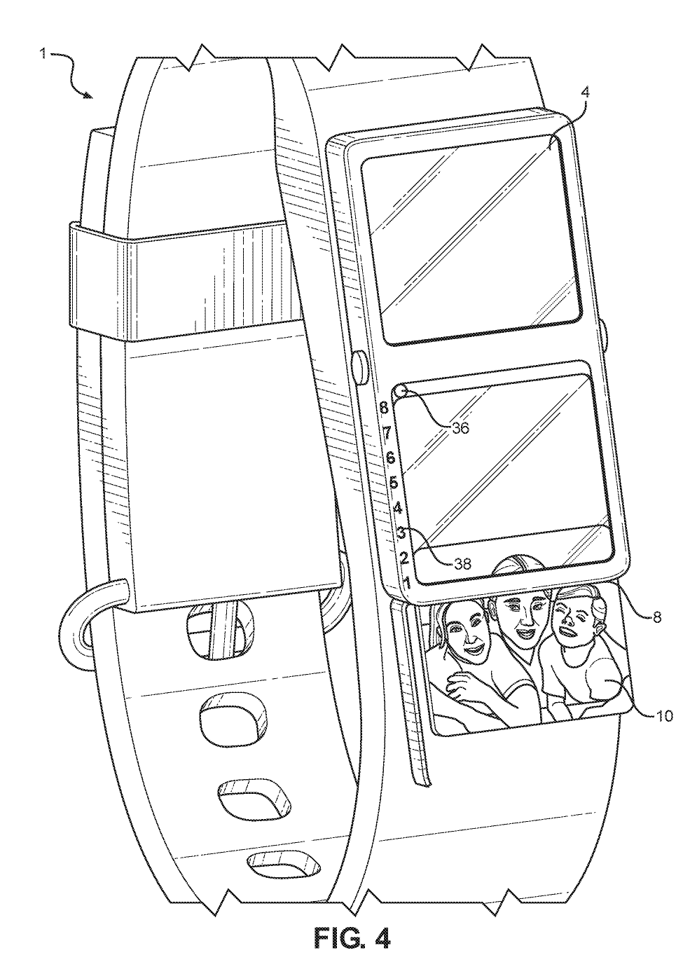

For the band version of the athletic tracking device 1, the Power photo (Pic) image 10 is manually inserted into the watch as best seen in FIG. 4.

The Power Pic Reveal & “Voltage Level”: For the watch version, best seen in FIG. 3, of the athletic tracking device 1, as a user is gradually making their goal, images 10 including the power pics (photos), videos (Flicks-like snapchats) and messages are revealed in various ways, by previously made selections on the app linked to the athletic tracking device 1. A Power Pic is revealed gradually as one gets closer to a goal, such as pieces of a whole pie uncovering the Power Photo, or revealed in a “ring-like” fashion or like paint being peeled back to reveal the full photo or video . . . the possibilities are endless. Through the app, users would be able to choose an image 10 including a single photo, video or message to reveal each day or multiple of these depending on the level of needed inspiration that the user desires.

To help keep users motivated through the day, users would have the option to reveal some images 10 including photos, videos or messages as they make progress and reach a certain percentage of their goal. In this case, the images 10 including power pic, videos, and messages would be like “volts” of power to help keep one inspired. Another option would be to choose to ONLY see the special Power Pic, video, or message images 10 once they reach their goal. These levels of options could be known as a “voltage level” and it could all be set and customized in the app.

This would allow the individual to customize how much inspiration or inspiration or daily volts one needs. The photos, videos, and message images 10 would rotate through in a specific order or are selected randomly, to reveal a surprise each day or when a goal is achieved. Bonus photos, video images, and voice message images 10 may be displayed when users surpass their goals, such as when they reach their goals five times in one week or go past their goals by 10%, with the parameters for the bonus items defined in the app.

For the band version of the athletic tracking device 1, as seen in FIG. 4, the photo image 10 is gradually revealed as the user pushes the front cover member 34 back one ridge 38 at a time as one slowly reaches their goal in order to reveal the “full” power photo image 10 that is helping them stay motivated.

The Power Pic—“Power Surge”: For the watch version of the athletic tracking device 1, when a person needs an extra inspirational boost of power, then they could request to see a uniquely dedicated photo, video, or message image 10 which could be known as a “Surge”, that is set up specifically via the app for days when an extra surge of motivation is needed.

Custom Mantra: For the watch version of the athletic tracking device 1, as seen in FIG. 3, a customized mantra can also be displayed as an image 10 constantly to keep one motivated. The mantra can be selected from a list of common motivational mantras or personally created using the app. Users would be able to choose the color and the font used for the words. For the band version of the athletic tracking device 1, as seen in FIG. 4, the mantra would be engraved on the front cover member 34.

The App: For the watch version of the athletic tracking device 1, using the app, users will be able to customize their goals just like other sport/fit watches. These would be tracked automatically using the pedometer 20 for monitoring the number of steps, the heart rate monitor 22, and the elevation monitor 24 for monitoring the number of flights of stairs that are climbed within a set period of time.

Power Pic “Friend Grid”: For the watch version of the athletic tracking device 1, friend Power Pics, videos, or message images 10 can be uploaded and displayed/sent to friends to be revealed once a friend achieves his or her goals or when one achieves their own goal and wants to share their success with friends. Also Power Pic “Surges” (photos, videos, or message images 10) can be sent to friends to motivate them to accomplish their goals. Group competitions would allow for Power Photos, videos, or message images 10 to be shared when the individual members of the group as each member reaches their goal and/or only when everyone in the group reaches their goals (multiple options). These options can be set up via the app. The Power Pic supports a “Power Grid” of friends to help each other surge on.

Power Pic “Community Sponsor Grid”: For the watch version of the athletic tracking device 1, as shown in FIG. 3, a powerful “grid” of companies could sponsor this fitness movement by offering rewards, such as coupons or prizes for certain health goal milestones. For example, if an individual walks over 100 miles in a month, a company would unlock a surprise gift photo, video or voice message image 10. It could be a coupon for saving money when making a purchase or a free gift, like a pair of running socks, sun block, sunglasses, etc. This surprise picture, video or voice message image 10 would have a barcode or coupon code that can be scanned or entered when claiming the prizes. Sponsoring businesses would set up the community goals via the Community Sponsorship Grid App, which would be a separate app for businesses to utilize. Individuals from the community would register for the sponsored goals using the Power Pic App.

Sponsoring businesses can establish the goals via the Community Sponsorship Grid App, where they can set up the goals and related prizes or rewards for accomplishing the goals. The goals the sponsor would set up might be walking so many miles a month or so many miles per year, etc. The app would have standard choices, such as expiration dates, number of uses for gifts/coupons, etc.”. This would be a “grid” of businesses helping to support health in the community by helping people find the power to reach their goals.

Power Pic “Sponsor Fundraising Grid”: Various businesses together would create a virtual “Power Grid” by raising funds and creating “power” in the community to improve health. For example, a sponsor may agree to donate 10 to the American Cancer Society or a community walking trail for every person that walks 100 miles within a specified time period. As a person using this device and app meets these goals, a related inspirational Power photo or video image 10 of the walking trail or maybe a young girl that has beat cancer can be displayed as inspiration. As part of the business/sponsorship fund raising or goal program, positive messages, advice, awareness, recommendations, and facts related to the fundraising event of goal will be shared to provide needed motivation. For example, when raising funds for an American Cancer Society event, when a user is 25% of the way towards the goal, a surprise pop-up photo, video, or voice message image 10 relating the fact that meditating for five minutes daily helps to decrease a person's changes of getting cancer can be displayed.

The Fit Pic Band Version: The band version of the athletic tracking device 1, as shown in FIG. 4, would be much simpler, with no automation. A Power photo image 10 would be hidden under a front cover member 34 which the user slides back slowly, one ridge 38 at a time as they work towards their goals, to reveal it. The front cover member 34 would have a mantra engraved on it for some extra inspiration. The front cover member 34 would likely slide under the part of the band where the logo is displayed. The photo image 10 would be added manually and is sealed to prevent it being damaged by moisture or perspiration. Individuals would choose a band with the appropriate number of ridges 38 on it that matches their individual goal. For example, if an individual is tracking the number of glasses of water he/she wants to drink a day, they would choose the wrist band 2 with 8 ridges 38. If the individual wanted to complete 10 sets of 10 exercises a day, they would choose a wrist band 2 with 10 notches. The band version would be much less expensive to purchase than any of the watch versions. A website could exist for an individual to choose the color, the number of ridges 38, and the personalized mantra to be engraved on the front cover member 34. Bands 2 with common popular mantras and a typical number of ridges 38 in various colors could also be sold.

Slider/Button Only Band: Another version of the athletic tracking device 1 band (likely the least-expensive version) would have one to four sliding trackers or buttons (like the sliding trackers on the watch version) with a photo/icon image of what is being tracked or the color of the band may represent what is being tracked, such as a blue band for tracking number of glasses of water. For example, one tracker/slider would have an icon of a “water droplet” to track the number of glasses of water consumed daily. Another slider/tracker would have an icon of piece of fruit to track the number of fruits consumed daily. Other trackers/sliders could be for tracking the number of veggies consumed daily (with an icon of a vegetable), or maybe the number of meditation breaks during the day (with an icon that stands for breathing or yoga). One band may only have a counter on it and be blue to represent that the user is tracking the number of glasses of water consumed. The possibilities of the various sliding trackers are endless. One version of the bands could be similar to collection of bangles (thin bands) and the user may have one bangle band for tracking water, another for tracking veggies, and the last one for tracking meditation breaks during the day, etc. User will be able to track their exercise, water, food, good deeds, miles, number of daily breaks or anything. A website would be used to select popular bands with “standard” slider/button trackers on them or users could create a custom band by choosing from an existing list of various slider trackers/buttons and band colors and designs. Lastly, they could also upload their own icon and choose the number of notches. This version would not have a personalized photo, but an icon photo image of what the user wants to track. No one enjoys logging into their phone and digging way down into an app 15 times a day to track their progress. These trackers will make it easy for individuals to track what is important to them. In addition, on the watch version, which has fruit and veggie trackers, the users could choose something different than the fruit and veggies trackers. The slider can be reset to zero by moving it back down to zero. The button could be reset to zero by holding down the button for a short period of time. The button version may also be reset via an app if it is tied to app where reports of the counter information could be created and an automatic reset time could be set.

The athletic tracking device 1 has a wrist band 2 which is adapted to releasably attach the athletic tracking device 1 to a user's wrist. The athletic tracking device 1 also has a tracking unit 4 attached to the wrist band 2 and which includes a main body 6 having a front panel portion 8 which is adapted to display one or more images 10. The images 10 are adapted to be individually and removably placed upon the front panel portion 8 depending on a chosen fitness goal. The tracking unit 4 is attached to the wrist band 2. The main body 6 has an interior volume 12 and a computer member 14 which is located within the interior volume 12 and is electronically connected to the front panel portion 8. The computer member 14 includes software applications adapted to track and provide information pertaining to fitness goals determined by the user. The front panel portion 8 may be a touchscreen enabling the user to select options relating to personalized fitness goals; wherein, said touch screen is configured to receive an electrical impulse from the touch of a user to input information relating to personalized fitness goals and to send that information to the computer member for analysis.

The computer member 14 is also adapted to provide portions of an image 10 to be displayed upon the front panel portion 8 depending upon the progress of the user reaching a chosen fitness goal. The main body 6 further includes a speaker member 16 electronically attached to the computer member 14 and is adapted to emit audio sounds depending on the progress of the user reaching a chosen fitness goal.

The images 10 to be displayed upon the front panel portion 8 includes video images and/or text messages as desired by the user. The main body 6 further includes a pedometer 20 electronically attached to the computer member 14 and is adapted to count the number of steps a user has taken during a chosen period of time and to send that information to the computer member 14 for analysis and display on the front panel portion 8.

The main body 6 further includes a microphone member 18 electronically attached to the computer member 14 and is adapted to receive audio sounds to be stored and used by the computer member 14. These audio sounds are adapted to be emitted by the speaker member 16 depending upon the progress of the user reaching a chosen fitness goal.

The main body 6 also includes a pedometer 20 electronically attached to the computer member 14 and is adapted to count the number of steps a user has taken during a chosen period of time and to send that information to the computer member 14 for analysis and display on the front panel portion 8. A heart rate monitor 22 is included in the main body 6 as well. The heart rate monitor 22 is also electronically attached to the computer member 14 and is adapted to monitor the heart rate of a user during a chosen period of time and to send that information to the computer member 14 for analysis and display on the front panel portion 8.

An elevation monitor 24 is also included in the main body 6 and is attached to the computer member 14 and is adapted to determine the elevation above sea level which the athletic tracking device 1 is located and to send that information to the computer member 14 for analysis and display on the front panel portion 8. The main body 6 also includes a receiver 26 electronically attached to the computer member 14 and is adapted to receive electronic signals and a transmitter 28 electronically attached to the computer member 14 adapted to send electronic signals such that the athletic tracking device 1 is adapted to communicate remotely with other computer systems.

The athletic tracking device 1 is adapted to receive information from companies and sponsors offering coupons and prizes for reaching chosen athletic goals. That information includes barcodes adapted to be displayed on the front panel portion 8 and used to retrieve the prizes from a merchant in possession of them.

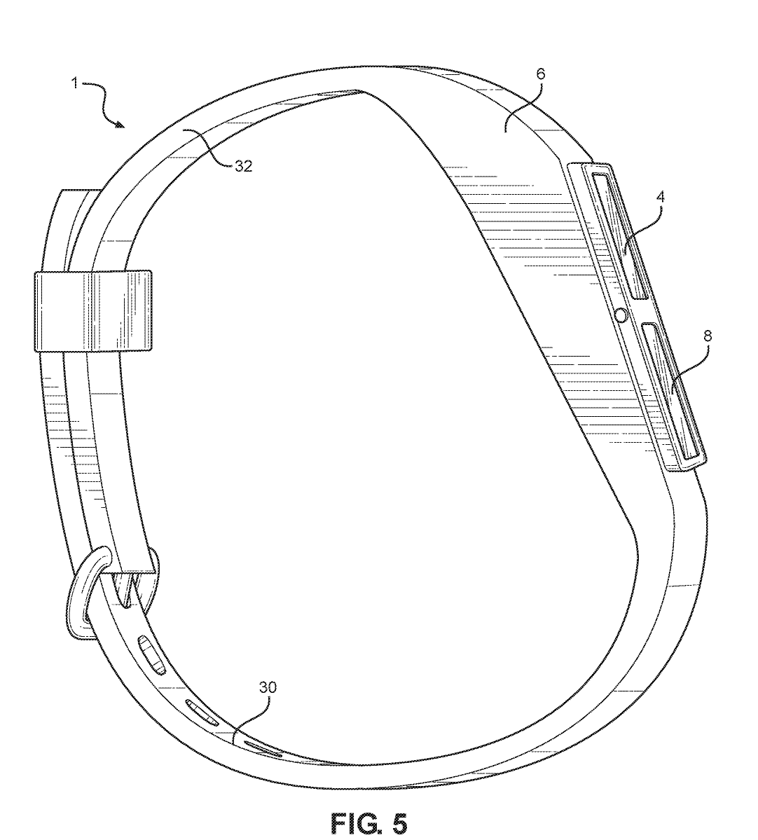

Referring to FIG. 5, the wrist band 2 of the athletic tracking device 1 has a first strap portion 30 attached to one side of the main body 6 and a second strap portion 32 attached to the opposite side of the main body 6. An end portion of the second strap portion 32 is removably attached to an end portion of the first strap portion 30 such that the athletic tracking device 1 is adapted to be removably and securely attached to the wrist of a user.

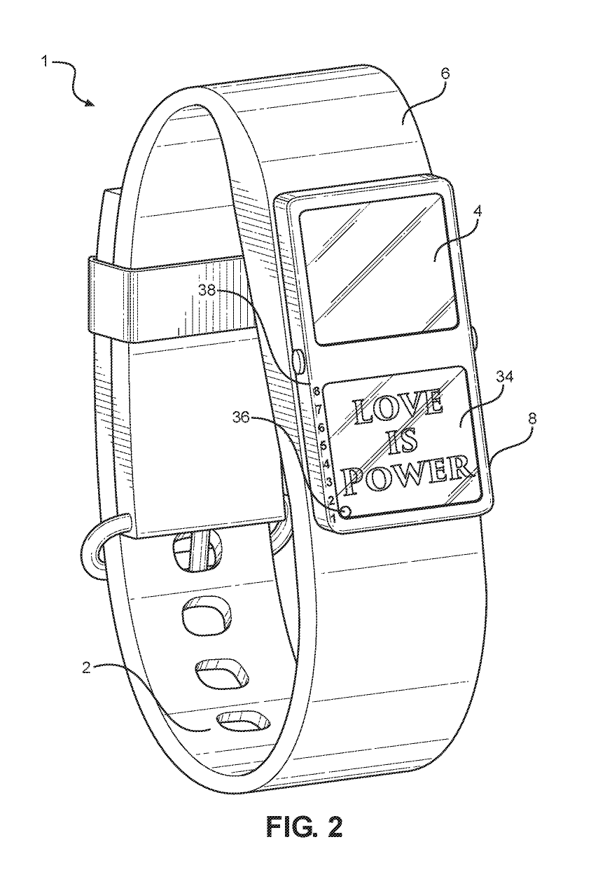

The athletic tracking device 1 also includes a front cover member 34, best seen in FIG. 2, adapted to be movably connected to the main body 6 and to selectively cover the front panel portion 8 depending upon a user reaching a chosen fitness goal. The front cover member 34 includes a handle member 36 which allows the user to slide the front cover member 34 with respect to the front panel portion 8 to reveal a chosen printed image in stages depending on the progress of a user reaching a chosen fitness goal.

The main body 6 further includes a plurality of ridges 38 adapted to removably retain the handle member 36 in predetermined locations to allow the user to slide the front cover member 34 with respect to the front panel portion 8 and reveal a chosen printed image 10 in stages depending on the progress of a user reaching a chosen fitness goal.

Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An athletic tracking device comprising:
a wrist band;
wherein said wrist band is adapted to releasably attach said athletic tracking device to a user's wrist;
a tracking unit including;
a main body including:
a front panel portion;
wherein said front panel portion is adapted to display images thereon;
an interior volume;
a computer member;
wherein said computer member is located within said interior volume, electronically connected to said front
panel portion, includes software applications adapted to track and provide information pertaining to fitness goals determined by said user, and is adapted to provide portions of an image to be displayed upon said front panel portion depending upon the progress of said user reaching a chosen fitness goal;
wherein said tracking unit is attached to said wrist band.

2. The athletic tracking device of claim 1, wherein said main body further includes a speaker member electronically attached to said computer member and is adapted to emit audio sounds depending upon the progress of said user reaching a chosen fitness goal.

3. The athletic tracking device of claim 2, wherein said main body further includes a microphone member electronically attached to said computer member and is adapted to receive audio sounds to be stored and used by said computer member; and wherein said audio sounds are adapted to be emitted by said speaker member depending upon the progress of said user reaching a chosen fitness goal.

4. The athletic tracking device of claim 1, wherein said image to be displayed upon said front panel portion includes video images.

5. The athletic tracking device of claim 1, wherein said image to be displayed upon said front panel portion includes text messages.

6. The athletic tracking device of claim 1, wherein said main body further includes a pedometer electronically attached to said computer member and is adapted to count the number of steps a user has taken during a chosen period of time and send that information to said computer member for analysis and display on said front panel portion.

7. The athletic tracking device of claim 1, wherein said main body further includes a heart rate monitor electronically attached to said computer member and is adapted to monitor the heart rate of a user during a chosen period of time and send that information to said computer member for analysis and display on said front panel portion.

8. The athletic tracking device of claim 1, wherein said main body further includes an elevation monitor electronically attached to said computer member and is adapted to determine the elevation above sea level the athletic tracking device is located at and send that information to said computer member for analysis and display on said front panel portion.

9. The athletic tracking device of claim 1, wherein said main body further includes a receiver electronically attached to said computer member and is adapted to receive electronic signals; and a transmitter electronically attached to said computer member and is adapted to send electronic signals, such that said athletic tracking device is adapted to communicate remotely with other computer systems.

10. The athletic tracking device of claim 9, wherein said athletic tracking device is adapted to receive information from companies and sponsors offering coupons and prizes for reaching chosen athletic goals.

11. The athletic tracking device of claim 10, wherein said information includes barcodes adapted to be displayed on said front panel portion and used to retrieve said prizes from a merchant in possession of said prizes.

12. The athletic tracking device of claim 1, wherein said wrist band further includes a first strap portion attached to one side of said main body; a second strap portion attached to an opposite side of said main body; wherein an end portion of said first strap portion is removably connectable to an end portion of said second strap portion such that said athletic tracking device is adapted to be removably and securely attached to a wrist of a user.

13. The athletic tracking device of claim 1 wherein said front panel portion is a touchscreen configured to receive an electrical impulse from the touch of a user to input information relating to personalized fitness goals and to send that information to said computer member for analysis.

14. An athletic tracking device comprising:
a wrist band;
wherein said wrist band is adapted to releasably attach said athletic tracking device to a user's wrist;
a tracking unit including;
a main body including:
a front panel portion;
wherein said front panel portion is adapted to display images thereon;
a plurality of printed images;
wherein said plurality of printed images are adapted to be individually and removably placed upon said front panel portion depending upon a chosen fitness goal;
a front cover member;
wherein said front cover member is adapted to be movably connected to said main body and selectively cover said front panel member depending on progress of a user reaching said a chosen fitness goal;
wherein said tracking unit is attached to said wrist band.

15. The athletic tracking device of claim 10, wherein said front cover member includes a handle member adapted to allow said user to slide said front cover member with respect to said front panel portion and reveal a chosen printed image in stages depending on the progress of a user reaching said chosen fitness goal.

16. The athletic tracking device of claim 11, wherein said main body further includes a plurality of ridges thereon each adapted to removably retain said handle member in predetermined locations to thereby allow said user to slide said front cover member with respect to said front panel portion and reveal a chosen printed image in stages depending on the progress of a user reaching said chosen fitness goal.

17. The athletic tracking device of claim 13, wherein said wrist band further includes a first strap portion attached to one side of said main body; a second strap portion attached to an opposite side of said main body; wherein an end portion of said first strap portion is removably connectable to an end portion of said second strap portion such that said athletic tracking device is adapted to be removably and securely attached to a wrist of a user.
